(19) United States
(12) Patent Application Publication
(10) Pub. No.: US 2013/0338234 A1
(43) Pub. Date: Dec. 19, 2013

(54) METHOD FOR DIRECT EXTRACTION AND CONCENTRATION OF NATURALLY-DERIVED ACTIVE COMPOUNDS

(71) Applicant: Radient Technologies Inc., Edmonton (CA)

(72) Inventors: Steven Splinter, North Vancouver (CA); Jocelyn J.R. Pare, Moncton (CA); Satyasagar Kadali, Burlington (CA)

(21) Appl. No.: 13/921,850

(22) Filed: Jun. 19, 2013

(30) Foreign Application Priority Data
Jun. 19, 2012 (CA) 2780578

(51) Int. Cl. A23L 1/025; A61K 31/122; A61K 31/047

(52) U.S. Cl. CPC A23L 1/025; A61K 31/047; A61K 31/122
USPC 514/691; 514/729

(57) ABSTRACT

The present invention relates to a method for the direct extraction or dispersion and concentration of naturally-derived active compounds into food-grade edible solvents or excipients directly usable in food, nutraceutical, pharmaceutical or cosmetic products without the use of synthetic organic solvents or organic solvents derived from petroleum or petrochemical products. The method involves a treatment of a multi-component biomass solvent system with microwave energy having a generally uniform high electric field intensity.

### CROSS REFERENCE TO RELATED APPLICATIONS

This application claims priority under 35 U.S.C. §119 to Canadian Application No. 2,780,578 filed on Jun. 19, 2012, the disclosure of which is incorporated by reference.

### FIELD OF THE INVENTION

This invention relates to a microwave treatment of substances for extracting and concentrating naturally-derived active compounds into food-grade edible solvents or excipients directly usable in food, nutraceutical, pharmaceutical or cosmetic products without the use of synthetic organic solvents or organic solvents derived from petroleum or petrochemical products, said treatment being carried out under non-equilibrium conditions.

### BACKGROUND OF THE INVENTION

Plants and natural products are the source of a significant array of bioactive compounds useful in numerous fields including, but not limited to, pharmaceuticals, nutraceuticals, functional foods and cosmetics. Because these useful compounds, often secondary metabolites, are present in the natural biological material in relatively low concentrations, the extraction, dispersion, recovery, concentration and purification of these commercially attractive natural ingredients is the focus of much industrial attention.

The common commercial methods of extraction include steam distillation, solvent extraction and more modern methods such as supercritical fluid extraction (SFE) using high pressure carbon dioxide and microwave-assisted extraction. Steam distillation is a very simple, albeit slow method of extraction used almost exclusively for relatively volatile products and is plagued by significant limitations with respect to fields of use. Solvent extraction is the most widely practised industrial method for obtaining natural ingredients. In their most traditional form, these methods tend to be complex, multi-step processes that involve the use of large amounts of organic solvents and multiple steps of selective solvent removal, separation and refinement. The organic solvents employed are often toxic and highly flammable. The processes therefore require expensive equipment to evaporate and recover as much of the solvent as possible and to remove most of the residual solvent from the final product and extracted spent solid residue. Complete removal of the organic solvent from the product and spent solid residue is, however, often impossible to guarantee. The presence of even trace amounts of toxic organic solvent residues in the product is a cause of concern with health-conscious consumers and has also limited the potential sale of the residual solids for human consumption and animal feedstock. The toxic and flammable nature of the organic solvents employed also necessitates expensive control systems and procedures to limit environmental impact and to ensure worker safety. Further, the need to remove those solvents gives rise to significant power requirements that in turn represent additional operating expenses in addition to further contribute significantly to environmental pollution derived from the production of said energy.

SFE is a technology that uses a pressurized liquefied gas (most commonly carbon dioxide) as the extraction solvent. Very high pressures are required. For example, carbon dioxide is only useful as a solvent at pressures in excess of 100 atmospheres. The high pressure requirement results in high capital equipment and operating costs and is the subject of significant operational safety concerns. Additionally, often the addition of so-called co-solvents is required to extract certain compounds into the pressurized solvent. These co-solvents can be environmentally undesirable and difficult to remove from the final product. Further, the gases used as supercritical fluids are often environmental pollutants. Still further, the use of such co-solvents is complicated by the fact that variations in contents as small as 0.1% can lead to non-reproducible results, thus affecting the quality of the final product.

In the prior art, processes have been disclosed which enable the extraction of active compounds such as antioxidants and spice components present in plants and other biomass into edible solvents such as vegetable oils and fats, principally by maceration or high pressure mixing. Typical procedures are disclosed in U.S. Pat. Nos. 2,571,867, 2,571,948, 3,732,111, 5,492,709 and 5,585,130. These processes use prohibitively high ratios of edible solvent in relation to the starting biomass, resulting in final products that are so weak with respect to the active components due to simple dilution that they cannot be used in many applications. In addition, these processes do not enable efficient transfer of the active components and require a great deal of processing time.

Several methods have been disclosed for simultaneously extracting and concentrating in a series of high-pressure mixing and mechanical pressing stages. These processes use relatively small amounts of vegetable oil as the solvent in an attempt to produce more concentrated final products. U.S. Pat. No. 4,681,769 to Bennett discloses a method for preparing flavoured and coloured spice oleoresin extracts by contacting ground spice with fortified edible oil containing spice elements and colour followed by pressure separation and blending the extracted spice residue with fresh oil to form the fortified edible oil for recycling in the extraction process. The process requires unduly long cycle times of up to 16 to 24 hours, adding significantly to the cost of the process, and is limited to narrow temperature and pressure ranges to avoid excessive yield losses and oxidative breakdown to the spice principles. U.S. Pat. Nos. 5,773,075, 6,013,304 and 6,074,687 to Todd claim a series of high temperature mixing and high-pressure filtration steps to produce concentrated herbal and spice extracts for food applications. The process is limited to high temperatures (55 to 230° C.) and high pressures (6000 to 30,000 psi) and so would not be suitable for the extraction of heat labile active components which decompose when exposed to high temperatures.

Other methods have been disclosed that are specific to particular compounds. U.S. Pat. No. 4,680,314 to Nonomura discloses a process for the production of a specific naturally-derived carotene-oil composition by direct extraction from algae. This process, however, is specific to wet algal concentrates containing as much as 90% water by weight. The presence of such a large quantity of water would result in inherently poor extraction efficiency due to fact that water would act as a barrier between the cells containing the carotenes and the oil solvent. This process also involves the use of other potentially toxic chemicals to provide critical pH adjustments during particular steps which could introduce undesired chemical residues. U.S. Pat. No. 7,557,146 to Sabio Rey discloses a process for preparing specific lycopene-enriched vegetable oil formulations by high speed mixing of a lycopene-containing vegetable source with vegetable oil at high temperature. The final product is weak in lycopene concentration, approximately 60 to 120 times weaker than similar products produced using normal solvent extraction procedures.

All of the above described prior art processes are based upon conventional extraction methods such as maceration or high-speed mixing and so are limited to the known extraction driving force of diffusion. The ability of such processes to successively concentrate active components to sufficiently high concentrations to be directly used for example in a pharmaceutical, nutraceutical, functional food or cosmetic formulations is severely restricted by the inherent reduction in the diffusional driving force as the extraction solvent becomes more concentrated in the active component and equilibrium conditions are approached. Adjustment of this equilibrium to allow continued extraction and concentration of active components can only be achieved by employing very high temperatures, which can lead to thermal degradation of many heat labile compounds, or by introduction of additional fresh, active-free solvent, which will only result in further reduction in the final concentration due to the simple dilution effect.

Microwave energy has been used in the prior art for extraction such as Ganzler et al. in Journal of Chromatography; 371, 1986: 299-306. However, this method applies microwave energy to a small amount of material immersed into strongly absorbing organic or petroleum-based solvents where the microwave energy is only providing an alternative heating source, without any microwave-specific heating characteristics. Further, such method does not provide any teachings with respect to concentration and dispersion or solution of final desired active ingredients.

U.S. Pat. No. 5,458,897 to Pare discloses a method of extraction of various substances from organic material by exposure to microwave energy. The organic material is surrounded by a medium relatively transparent to microwaves, so that microwave energy is preferentially absorbed by the microwave absorbing constituents of the organic material, causing differential heating of the material over the medium sufficient to disrupt the structure of the organic material releasing the desired substances. Volatile oils and other substances can be extracted according to this method without significant heating. There are no teachings about extraction into non organic or non-petroleum based solvents. Further, there are no teachings about the concentration or dispersion of bioactive compounds directly into a non-organic, non-petroleum based solvent.

U.S. Pat. No. 5,377,426 to Pare discloses a method of generating volatiles from liquid or solid materials enhanced by exposure to microwave radiation in the absence of solvent. Microwave energy according to this prior art method is absorbed preferentially by the liquid or solid material over generated gaseous volatiles allowing substantially complete volatilization of the desired substances. There are no teachings about extraction into non organic or non-petroleum based solvents. Further, there are no teachings about the concentration or dispersion of bioactive materials directly into the non-organic, non-petroleum based solvent.

Microwave energy has been shown to successfully enhance the extraction of desired components more quickly and efficiently than prior art methods, with greater selectivity and less product degradation. These methods rely on the different capacity of various materials to absorb microwave energy based on its dielectric properties. For example, U.S. Pat. No. 6,061,926 to Pare teaches that high intensity electric field can lead to non-equilibrium conditions that in turn enhance various chemical processes. However, to date there has been no method for the direct extraction of bioactive compounds directly into a non-organic, non-petroleum based solvent. Further there has been no method about concentrating or dispersing bioactive materials directly into non-organic, non-petroleum based solvent.

Final concentrated or purified active compounds are often diluted or dispersed and standardized into an oil, fat or other lipid-based excipient or emulsifier to a desired concentration to be used for example in a pharmaceutical, nutraceutical, functional food or cosmetic formulation ready for sale and use. Prior art processes do not allow for fortification of the active components to a sufficiently high concentration to be directly used for example in a pharmaceutical, nutraceutical, functional food or cosmetic formulation. Thus, there is a need for direct extraction of bioactive components into a final dispersion or solution medium. Further, there is a need for the concentration of said bioactive components to the final desired concentration in said dispersion or solution medium without other intermediate steps and devoid of the requirement to use potentially toxic synthetic organic solvents or organic solvents derived from petroleum or petrochemical products. Hence, providing microwave-induced non-equilibrium conditions that can favour enhanced concentration is highly desirable. Such a method would be of significant commercial value.

### SUMMARY OF THE INVENTION

An embodiment of one aspect of the invention is to provide a method for direct extraction or dispersion and concentration of bioactive compounds from natural biological materials into food-grade edible solvents or excipients which are typically used to standardize the resulting extract to a desired concentration of active ingredient and that is completely free of synthetic organic solvents or organic solvents derived from petroleum or petrochemical products or highly flammable solvents.

Another embodiment of one aspect of this invention is to provide a method for direct extraction or dispersion of bioactive compounds from natural biological materials that is economical and environmentally friendly.

A further embodiment of one aspect of this invention is to provide a method for extraction or dispersion of bioactive compounds from natural biological materials that does not require expensive multiple steps of selective solvent removal, separation and refinement.

A still further embodiment of one aspect of this invention is to provide a method for direct extraction or dispersion and successive concentration of bioactive compounds from natural biological materials in food grade edible solvents to a desired concentration to be used for example in a pharmaceutical, nutraceutical, functional food or cosmetic formulations ready for sale and use.

Another embodiment of one aspect of this invention is to provide a method for direct extraction or dispersion of bioactive compounds from natural biological materials into food-grade edible solvents or excipients that minimizes contact time with the extractant and avoids excessive degradation of sensitive compounds and provides for products of improved stability.

Another embodiment of one aspect of this invention is to provide a method for preparing natural ingredients for pharmaceutical, nutraceutical, food and cosmetic applications that are more beneficial than those prepared by mixing purified bioactive compounds with food-grade edible solvents or excipients due to additional content of other phytochemicals that are extracted for the biological material which may have beneficial synergistic effects with the primary bioactive compounds and which are not degraded.

Another embodiment of one aspect of this invention is to provide a method for preparing a natural ingredient and a residual extracted spent solid that is completely free of residual synthetic organic solvents or organic solvents derived from petroleum or petrochemical products or other adulterants.

Another embodiment of one aspect of this invention is to provide a method for preparing a natural ingredient and a residual extracted spent solid that have reduced microbial counts.

Another embodiment of one aspect of this invention is to provide a method for preparing a residual extracted spent solid that has not undergone any chemical treatment and can be used directly or to produce other products for use in human or animal consumption.

Another embodiment of one aspect of this invention is to provide a method for preparing a residual extracted spent solid that has had its microstructure disrupted and so has improved bio-availability.

Yet another embodiment of one aspect of this invention is to provide means to enhance the electric field component of the microwaves so that the treatment of the natural materials is performed under non-equilibrium conditions.

One embodiment of one aspect of this invention provides a method for the direct extraction of bioactive substances into non organic, non-petroleum based solvent, comprising the steps of:
providing a source of microwave energy having a generally uniform high electric field;
providing an extraction or a dispersion system to be treated comprising a multi-component biomass capable of absorbing a portion or all of said high electric field microwave energy in contact with a non-toxic, non-synthetic organic, non-petroleum based solvent capable of solubilizing or dispersing components making up said biomass;
optionally providing a means to agitate said extraction or said dispersion system;
exposing said extraction or said dispersion system to said high electric field microwave energy, at an electric field intensity such that said biomass will release components making up said biomass into said non-organic, non-petroleum based solvent;

Another embodiment of one aspect of this invention comprises the steps of:
providing a source of microwave energy having a generally uniform high electric field;
providing an extraction or a dispersion system to be treated comprising a multi-component biomass capable to absorb a portion or all of said high electric field microwave energy in contact with a non-toxic, non synthetic organic, non-petroleum based solvent capable of solubilizing or dispersing components making up said biomass;
optionally providing a means to agitate said extraction or dispersion system;
exposing said extraction or dispersion system to said high electric field microwave energy, at an electric field intensity such that said biomass will release components making up said biomass into said non-organic, non-petroleum based solvent; and
continuing treatment of said extraction or dispersion system for such a time that sufficient high electric field intensity microwave energy has been absorbed by said biomass to induce the release of components making up said biomass at a level equivalent or superior to that observed under thermal equilibrium conditions.

Another embodiment of one aspect of the invention relates to an improvement to the process, the improvement being the use of a chemical susceptor to induce the release of bioactive components, and wherein the improvement comprises the steps of:
providing a source of microwave energy having a generally uniform high electric field;
providing an extraction or a dispersion system to be treated comprising a multi-component biomass capable to absorb a portion or all of said high electric field microwave energy in contact with a non-toxic, non synthetic organic, non-petroleum based solvent capable of solubilizing or dispersing components making up said biomass;
providing a chemical susceptor to said extraction or dispersion system and more specifically into said multi-component biomass forming part of said extraction or dispersion system so as to further increase the localized heating into said multi-component biomass forming part of said extraction or dispersion system;
optionally providing a means to agitate said extraction or dispersion system;
exposing said extraction or dispersion system to said high electric field microwave energy, at an electric field intensity such that said biomass will release components making up said biomass into said non-organic, non-petroleum based solvent; and
continuing treatment of said extraction or dispersion system for such a time that sufficient high electric field intensity microwave energy has been absorbed by said biomass to induce the release of components making up said biomass at a level equivalent or superior to that observed under thermal equilibrium conditions.

It will be evident to those skilled in the art that a high electric field as referred to in some embodiments of one aspect of this invention can be obtained through the judicious use of mechanical microwave-guiding devices or through the use of appropriate chemical components that do not interfere with the treatment itself nor with the products, or the combination of said microwave-guiding devices and said chemical substances, such that the treatment is performed under non-equilibrium conditions.

It will be obvious to those skilled in the art that step (c) of the latter aspect of this invention may bring about reductions in time periods required to effect the treatment, thus leading to significant energy savings.

### BRIEF DESCRIPTION OF THE DRAWINGS

FIG. 1 is a graph showing results obtained for the direct microwave-assisted extraction and concentration of lutein and zeaxanthin from natural paprika biomass into corn oil.

FIG. 2 is a further graph illustrating results obtained for the direct microwave-assisted extraction and concentration of lutein and zeaxanthin from natural marigold biomass into corn oil.

FIG. 3 is a further graph illustrating results obtained for the direct microwave-assisted extraction and concentration of total carotenoids, including astaxanthin, from natural microalgae biomass into safflower oil.

### DETAILED DESCRIPTION OF THE INVENTION

The features and other details of the invention will now be particularly described.

The methods of the invention begin with providing a source of microwave energy having a generally uniform high electric field. It will be evident to those skilled in the art that a generally uniform high electric field can be achieved by employing microwave energy concentrating means such as waveguides at controlled frequency and energy levels. A generally uniform high electric field in the form of microwave energy density applied for a given mass of microwave absorbers over a given volume can be controlled and managed using a focused microwave applicator and waveguide.

A preferred embodiment of one aspect of this invention is to provide a source of microwave energy having a generally uniform electric field by making use of focused microwaves by means of waveguides.

A more preferred embodiment of one aspect of this invention is to make use of appropriate microwave devices that allow for the maintenance of standing waves over localized volumes.

Another preferred embodiment of one aspect of this invention is to make use of appropriate chemical entities known as microwave susceptor to further enhance the electrical field component of the microwaves without applying further overall energy.

Yet a more preferred embodiment of one aspect of this invention is to provide means to control the exposition period to microwaves while further enhancing the intensity of the electrical field component of the microwaves to levels higher than those conventionally required, thus still reducing the overall energy applied to the system, thus further reducing the operational costs.

It will be evident to those skilled in the art that the latter embodiment of one aspect of this invention can further be improved in terms of operational efficiency by providing means to cool the treatment cavity and by providing control means for said cooling features.

It has been found that when a given mass of a multi-component biomass capable of absorbing a portion or all of the high electric field microwave energy is exposed to said high electric field microwave energy, the energy absorbed exceeds the ability of the system to dissipate the energy as thermal energy and, as a result, a non-steady thermodynamic state is created within the system. It will evident to those skilled in the art that the rate at which microwave energy can be deposited into a liquid system is of the order of 1,000,000 times faster than the system can dissipate such energy by thermal diffusion in the liquid medium and of the order of 1,000 times faster than the system can dissipate such energy by conventional relaxation processes in the liquid medium.

It has been further found that by continuing the microwave treatment for such a time that sufficient high electric field intensity energy has been deposited into the system, an effect, such as the extraction of bioactive compounds from a biomass into a liquid medium, occurs at a rate much faster than would otherwise be expected due to the energy applied and that the release of said bioactive compounds occurs at a higher level than would otherwise be expected under thermal equilibrium conditions.

It will be evident to those skilled in the art that any wavelength within the microwave spectrum capable of being absorbed to some extent by a component of the system can be used. A typical frequency would be in the range of about 20 to 900,000 MHz. A preferred embodiment of one aspect of this invention makes use of microwave energy at an approximate frequency of 2450 MHz. Another preferred embodiment of one aspect of this invention makes use of microwave energy at an approximate frequency of 915 Mhz. The overall microwave power or energy to be applied may be selected and varied according to the nature of the system of interest.

This invention provides methods for direct extraction or dispersion and concentration of bioactive compounds from natural biomass materials into food-grade edible solvents or excipients. The types of biomass materials applicable to this invention include any organic matter containing bioactive compounds. Organic matter is herein meant to include any matter containing or derived from living organisms, including but not limited to plants, algae, fungi, and bacteria.

In a preferred embodiment of one aspect of this invention the organic matter will be comprised of a multi-component biomass capable of absorbing a portion or all of the high electric field energy applied to it.

While the biomass material to be treated may be fresh, it is preferably dried prior to extraction or dispersion, in order to minimize potential decomposition and improve shelf-life. In a preferred embodiment of one aspect of this invention the biomass material is dried to a residual moisture content of less than 20% by weight. In yet a more preferred embodiment of one aspect of this invention the biomass material is dried to a residual moisture content of less than 10% by weight.

The biomass material to be treated is preferably manipulated prior to treatment to increase the surface area. Methods to manipulate the biomass material to increase the surface area will be known to those skilled in the art and include for example cutting, chopping, crushing or milling.

The solvent may be any suitable non-organic, non-petroleum based solvent in which the active is soluble or can be dispersed and which is authorized for use by regulations in the country to which the final product is to be marketed. The solvent may be used as a carrier for the active components when formulated into a product and may be selected according to the intended use of the product to impart for example desired physical or chemical properties to the product such as odour or health-related properties. In a preferred embodiment of aspect of this invention, the solvent will be an edible or food-grade solvent or an excipient or emulsifier typically used to standardize active compounds in pharmaceutical, nutraceutical, functional food, or cosmetic formulations.

In a preferred embodiment of one aspect of this invention, the solvent is selected from the group of corn oil, safflower oil, borage oil, flax oil, canola oil, cottonseed oil, soybean oil, olive oil, sunflower oil, mono-, di- and triglycerides, lecithin, limonene, essential oils of spices, herbs or other plants, fish oil, glycerol, glycols, or any other, or mixtures thereof.

In a more preferred embodiment of one aspect of this invention, the solvent is a vegetable oil that has been obtained by simple mechanical expression, without requiring organic or petroleum based solvents for processing.

The ratio of non-organic, non-petroleum based solvent to biomass in the system to be treated can range from about 0.5 to 20 kg/kg. It will be evident to those skilled in the art that this ratio should be maintained as low as possible in order to maximize the concentration of active components in the final product by minimizing simple dilution effects. This low ratio also brings about further operational costs reductions.

In a preferred embodiment of one aspect of this invention, the solvent to biomass ratio is between about 1 to 5 kg/kg.

Following direct extraction or dispersion, the residual extracted biomass solids can be separated from the active-containing solvent by any suitable means as are known in the art, including but not limited to centrifugation, filtration, gravity separation or settling. It will be evident to those skilled in the art that the separation method employed should be capable of removing as much of the solvent from the residual extracted biomass solids as possible. The extract may be further filtered or centrifuged to provide a finished product free of particulate solids.

In a preferred embodiment of one aspect of this invention, the treatment method involves a plurality of treatment and separation stages wherein the active-containing solvent following separation from one stage is used as the starting solvent for a second treatment stage utilizing fresh biomass material so as to further concentrate the bioactive compounds in the non-organic, non-petroleum based solvent.

In a still further preferred embodiment of one aspect of this invention, the plurality of treatment and separation stages is carried out so as to effect a successive concentration of bioactive compounds in the solvent to a sufficiently high concentration as to be used directly in pharmaceutical, nutraceutical, functional food or cosmetic formulations. In a preferred embodiment, the successive staged treatments are carried out in a counter-current flow pattern as is known in the art.

In another embodiment of one aspect of this invention, an improvement is made to the process, wherein a chemical susceptor is added to the treatment system to further increase the localized heating in the multi-component biomass forming part of the treatment system and so induce release of the bioactive components from said biomass.

In yet another preferred embodiment of one aspect of this invention, a chemical susceptor is added to the treatment system in order to significantly reduce the time period required for the treatment. The chemical susceptor may be a compound of natural origin.

It will be evident to those skilled in the art that the selection of such a chemical susceptor will be based upon the relative differences in permittivity, or static dielectric constant, and loss factors between the susceptor and the biomass to be treated, or between the susceptor and the solvent system used to effect the extraction, separation, or concentration treatment. It will be further evident to those skilled in the art that such susceptor will also be selected according to its neutrality to the system used, thus avoiding the potential to effect any changes to the system, and further avoiding potential contamination of said system.

In a preferred embodiment of one aspect of this invention the chemical susceptor is an inorganic carbide such as for example, silicon carbide or tungsten carbide. These compounds are mentioned merely as examples to illustrate the type of chemicals and do not limit the nature of such chemical entity to be used as said susceptor.

The treatment may be carried out in continuous or in a batch fashion. The treatment may be carried out under agitation using any suitable means known in the art.

In one embodiment of one aspect of this invention, antioxidants may be added to the biomass or solvent prior to treatment to minimize potential degradation of the bioactive compounds or the solvent. Preferably, the antioxidant will be an edible antioxidant and be an approved food-grade additive such as ascorbic acid, tocopherol, BHA, BHT, etc. More preferably, the antioxidant is a naturally-derived plant-based antioxidant such as extracts of rosemary, sage or thyme.

In a preferred embodiment of one aspect of this invention, the treatment is carried out under reduced oxygen partial pressure to reduce potential oxidative degradation of the bioactive compounds and possible degradation of the solvent. In a more preferred embodiment, the treatment is carried out under nitrogen or under another inert gas blanket.

### EXAMPLES

### Example 1

This example demonstrates the inventive nature of the method disclosed to directly extract the important carotenoid pigments lutein and zeaxanthin from paprika powder into a solvent comprised of natural corn oil.

Carotenoids are natural fat soluble pigment molecules present in a wide variety of plants and other natural products. Dietary carotenoids have been shown to provide a range of health benefits and to decrease risk of disease, primarily by acting as strong antioxidants, quenching the excess energy of free radicals generated by life's processes. The most important carotenoids include beta-carotene, lycopene, lutein, zeaxanthin and astaxanthin. Lutein and zeaxanthin occur in their greatest concentrations in the human eye, with lutein concentrated in the retina and zeaxanthin concentrated in the macula. Supplementation with lutein and zeaxanthin increases macular pigment density, protecting against oxidative damage to eye tissues.

Sweet paprika was chosen as a natural biomass source of lutein and zeaxanthin for illustrative purposes. The paprika biomass was analyzed for lutein and zeaxanthin by standard High Performance Liquid Chromatography (HPLC) methods and found to contain a total of 0.12% w/w total lutein+zeaxanthin. Approximately 25 g samples of dried, finely ground paprika were contacted with 250 mL of corn oil and gently agitated in a modified laboratory-scale microwave extractor and subjected to various microwave power levels at 2450 Mhz for 5 minutes. The mixture was then gently agitated for 25 minutes following microwave exposure. To demonstrate the effect of the high electric field microwaves, additional experiments were conducted in the identical equipment but without microwave exposure. In these experiments the mixture was gently agitated for times of 30 min, 4 h and 24 h. Following all experiments, the extracted solids were then separated from the extract solution by filtration. Samples of the corn oil were quantitatively analyzed for lutein and zeaxanthin content by HPLC. The extraction efficiency was determined as the percent total recovery of available lutein+zeaxanthin from the starting biomass. Table 1 shows the concentration of lutein+zeaxanthin in the corn oil after each experiment and the calculated percent recovery of available lutein+zeaxanthin.

TABLE 1
Direct extraction of lutein and zeaxanthin from sweet paprika into corn oil

| | | Concentration | | |
|---|---|---|---|---|
| | | of Lutein + | | |
| Experi- | | Zeaxanthin (HPLC) | | Total % |
| ment | Treatment Conditions | % w/w | mg/mL | Recovery |
| 1 | No microwaves/30 min | 0.0048% | 0.0432 | 36% |
| 2 | No microwaves/4 h | 0.0052% | 0.0468 | 39% |
| 3 | No microwaves/24 h | 0.0065% | 0.0585 | 49% |
| 4 | 300 Watt Microwaves/5 min | 0.0056% | 0.0504 | 42% |
| 5 | 600 Watt Microwaves/5 min | 0.0091% | 0.0819 | 68% |
| 6 | 900 Watt Microwaves/5 min | 0.0127% | 0.1143 | 95% |

The use of this invention, in this example, shows the effect of field intensity and the benefits derived from using this embodiment of one aspect of the invention with regard to extraction efficiency.

### Example 2

Approximately 25 g samples of dried, finely ground paprika were contacted with varying amounts of corn oil and gently agitated in a modified laboratory-scale microwave extractor and subjected to focused 2450 Mhz microwave energy at 600 Watts for 5 minutes. The mixture was then gently agitated for 25 minutes following microwave exposure. Following the experiments, the extracted solids were then separated from the extract solution by filtration. Samples of the corn oil were quantitatively analyzed for lutein and zeaxanthin content by HPLC. The extraction efficiency was determined as the percent total recovery of available lutein+zeaxanthin from the starting biomass. Table 2 shows the concentration of lutein+zeaxanthin in the corn oil after each experiment and the calculated percent recovery of available lutein+zeaxanthin.

TABLE 2
Direct microwave-assisted extraction of lutein and zeaxanthin from sweet
paprika into corn at 600 Watts microwave power for 5 min.

| | Solvent to | Concentration of Lutein + | | |
|---|---|---|---|---|
| | Solids ratio | Zeaxanthin (HPLC) | | Total |
| Experiment | L/kg | % w/w | mg/mL | Recovery % |
| 1 | 10 | 0.0091% | 0.0819 | 68% |
| 2 | 5 | 0.0206% | 0.1854 | 77% |
| 3 | 2.5 | 0.0508% | 0.4572 | 96% |

The use of this invention, in this example, shows the effect of field intensity and the benefits derived from using this embodiment of one aspect of the invention with regard to extraction efficiency and with regard to treatment costs as evidenced by the reduction in solvent usage.

### Example 3

This example demonstrates a preferred embodiment of one aspect of the method disclosed to directly extract and successively concentrate a bioactive compound into a food grade edible solvent to a concentration that can be used in a nutraceutical formulation.

Epidemiological evidence suggests that around 5 to 6 mg/day of total lutein+zeaxanthin has a beneficial effect on eye health. The desired concentration for direct use in 1 mL sized gelcaps would then be in the range of 2.5 mg of active per mL so that a daily dose could be easily administered by two such gelcaps.

Approximately 100 g of dried, finely ground paprika was contacted with 250 mL of corn oil and gently agitated in a modified laboratory-scale microwave extractor and subjected to focused 2450 MHz microwave energy at 600 Watts for 5 minutes. The mixture was then gently agitated for 25 minutes following microwave exposure and the extracted solids were then separated from the extract solution by filtration. Samples of the corn oil were quantitatively analyzed for lutein and zeaxanthin content by HPLC. The corn oil extract fortified with lutein and zeaxanthin was then used as the starting solvent for a second treatment stage utilizing fresh paprika biomass material so as to further concentrate the lutein and zeaxanthin in the corn oil solvent.

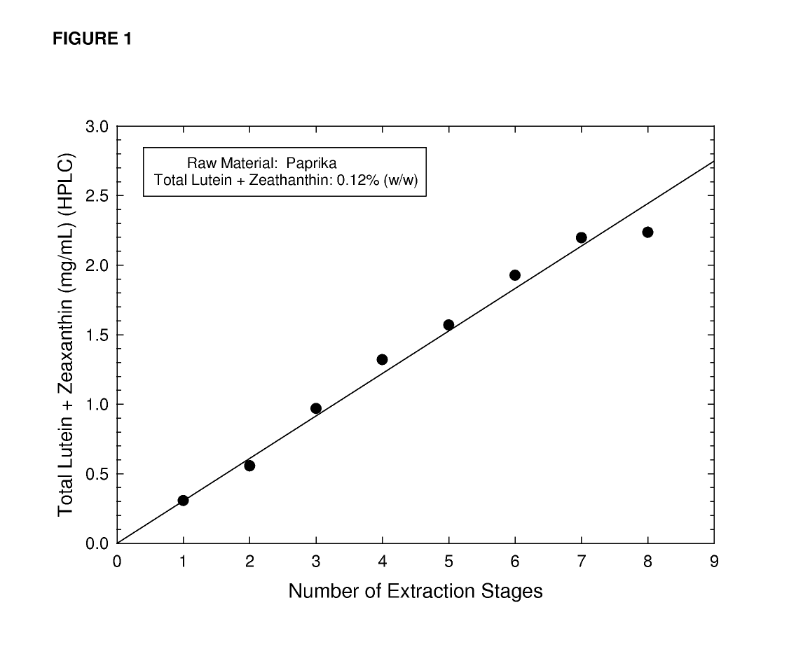

This procedure was repeated for a total of eight treatment stages. FIG. 1 shows the total concentration of lutein+zeaxanthin in the corn oil after each treatment stage as determined by HPLC.

The use of this invention, in this example, shows the effect of field intensity and the benefits derived from using this embodiment of one aspect of the invention, namely providing non-equilibrium conditions with regard to the direct extraction, dispersion and concentration of naturally-derived active compounds into food-grade edible solvents or excipients directly usable in food, nutraceutical, pharmaceutical or cosmetic products without the use of synthetic organic solvents or organic solvents derived from petroleum or petrochemical products.

### Example 4

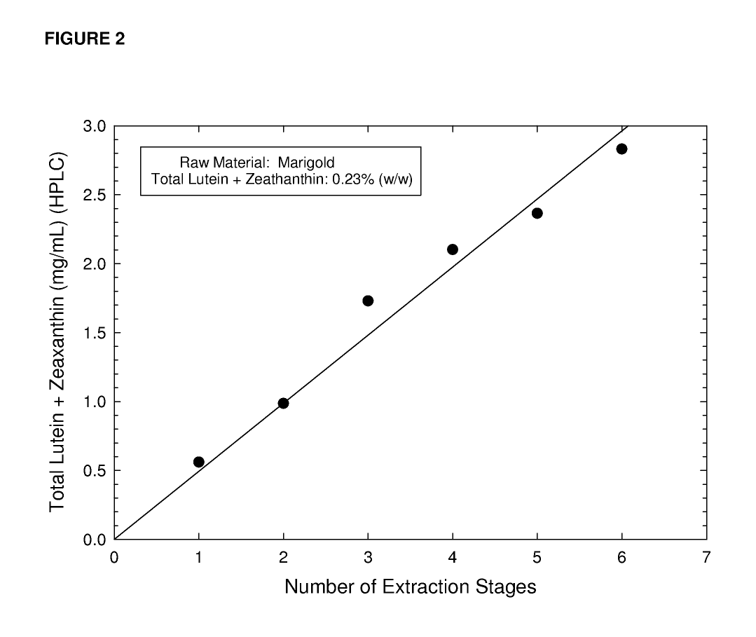

Dried, finely ground marigold meal was analyzed for lutein and zeaxanthin by standard HPLC methods and found to contain a total of 0.23% w/w total lutein+zeaxanthin. The same conditions as were employed in example 3 was used to successively concentrate lutein and zeaxanthin from the marigold meal into corn oil over six treatment stages. FIG. 2 shows the total concentration of lutein+zeaxanthin in the corn oil after each treatment stage as determined by HPLC.

The use of this invention, in this example, shows the effect of field intensity and the benefits derived from using this embodiment of one aspect of the invention, namely providing non-equilibrium conditions with regard to the direct extraction, dispersion and concentration of naturally-derived active compounds into food-grade edible solvents or excipients directly usable in food, nutraceutical, pharmaceutical or cosmetic products without the use of synthetic organic solvents or organic solvents derived from petroleum or petrochemical products.

### Example 5

This example demonstrates the inventive nature of the method disclosed to directly extract the important carotenoid astaxanthin from dried microalgae into a solvent comprised of natural safflower oil. Natural astaxanthin is used as a food supplement for human, animal and aquaculture consumption. It has been shown to have potent antioxidant activity and so may protect body tissues from oxidative damage. Research suggests that supplementation with astaxanthin may be beneficial in immune, cardiovascular, inflammatory and neurodegenerative diseases.

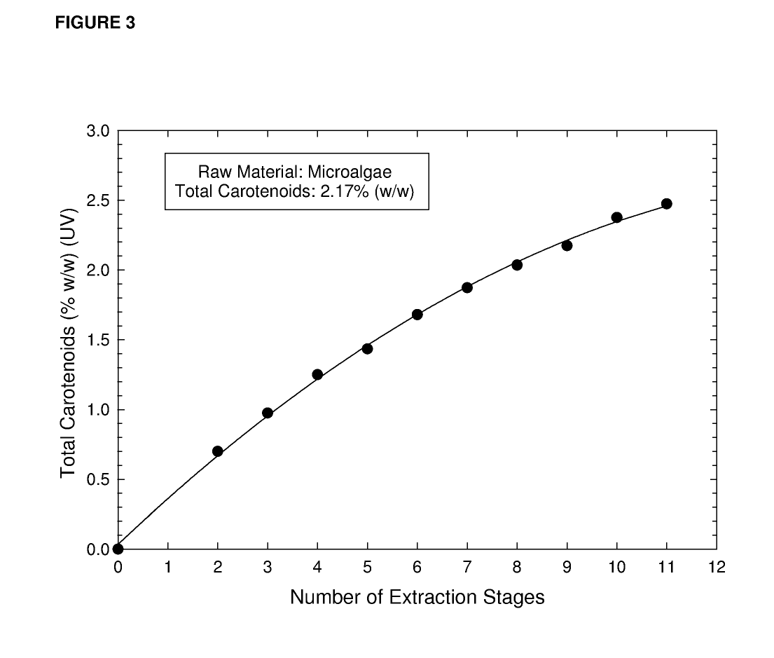

Dried, ground haematoccocus microalgae was analyzed by Ultraviolet Spectroscopy (UV) analysis and found to contain 2.17% w/w total carotenoids. Approximately 100 g of dried, finely ground microalgae was contacted with 500 mL of safflower oil and gently agitated in a modified laboratory-scale microwave extractor and subjected to focused 2450 Mhz microwave energy at 300 Watts for 5 minutes. The mixture was then gently agitated for 25 minutes following microwave exposure and the extracted solids were then separated from the extract solution by filtration. Samples of the safflower oil were quantitatively analyzed for total carotenoids by UV analysis. The safflower oil extract fortified with astaxanthin was then used as the starting solvent for a second treatment stage utilizing fresh microalgae biomass material so as to further concentrate the astaxanthin in the safflower oil solvent. This procedure was repeated for a total of eleven treatment stages. FIG. 3 shows the total concentration of carotenoids in the safflower oil after each treatment stage as determined by UV analysis. The safflower oil extract fortified with astaxanthin from the final treatment stage was analyzed for astaxanthin concentration by HPLC and found to contain 2.1% w/w (approximately 19 mg/mL) of astaxanthin.

The use of this invention, in this example, shows the effect of field intensity and the benefits derived from using this embodiment of one aspect of the invention, namely providing non-equilibrium conditions with regard to the direct extraction, dispersion and concentration of naturally-derived active compounds into food-grade edible solvents or excipients directly usable in food, nutraceutical, pharmaceutical or cosmetic products without the use of synthetic organic solvents or organic solvents derived from petroleum or petrochemical products.

We claim:

1. A method for the direct extraction or dispersion of bioactive substances into non organic, non-petroleum based solvent, comprising the steps of:
a) providing a source of microwave energy having a generally uniform high electric field;
b) providing an extraction or dispersion system to be treated comprising a multi-component biomass capable to absorb a portion or all said high electric field microwave energy in contact with a non-organic, non-petroleum based solvent capable to solubilize or disperse components making up said biomass;
c) optionally providing means to agitate said extraction or dispersion system;
d) exposing said extraction or dispersion system to said high electric field microwave energy, at an electric field intensity such that said biomass will release components making up said biomass into said non-organic, non-petroleum based solvent;
e) continuing treatment of said extraction or dispersion system for such a time that sufficient high electric field intensity microwave energy has been absorbed by said biomass to induce the release of components making up said biomass at a level equivalent or superior to that observed under thermal equilibrium conditions.

2. The method as defined in claim 1 where a further substance is added to said extraction or dispersion system and more specifically into said multi-component biomass forming part of said extraction or dispersion system so as to further increase the localized heating into said multi-component biomass forming part of said extraction or dispersion system.

3. The method as defined in claim 1 where a further substance is added to said extraction or dispersion system and more specifically into said multi-component biomass forming part of said extraction or dispersion system so as to further increase the localized heating into said multi-component biomass forming part of said extraction or dispersion system and to reduce the treatment time.

4. The method as defined in claim 1 including the step of recovering at least one component or product from said extraction or dispersion system after the equilibrium of said extraction or dispersion system has been preferentially shifted to said non-equilibrium state.

5. The method as defined in claim 1 where so produced extract or dispersion is used as the non-organic, non petroleum based solvent to further concentrate the quantity of components of biomass into said non-organic, non petroleum-based solvent

6. The method as defined in claim 2 wherein the chemical susceptor is a compound of natural origin.

7. The method as defined in claim 2 wherein the chemical susceptor is a compound that does not interact with any of the substances making up said extraction or dispersion substance.

8. The method as defined in claim 7 where the chemical susceptor is an inorganic carbide.

9. A method for the direct extraction or dispersion of bioactive substances into non organic, non-petroleum based solvent, comprising the steps of:
a) providing a source of microwave energy having a generally uniform high electric field;
b) providing an extraction or dispersion system to be treated comprising a multi-component biomass capable to absorb a portion or all said high electric field microwave energy in contact with a non-organic, non-petroleum based solvent capable to solubilize or disperse components making up said biomass;
c) providing a chemical susceptor to said extraction or dispersion system so as to further increase the field in said extraction system and more specifically into said multi-component biomass forming part of said extraction or dispersion system;
d) optionally providing means to agitate said extraction or dispersion system;
e) exposing said extraction or dispersion system to said high electric field microwave energy, at an electric field intensity such that said biomass will release components making up said biomass into said non-organic, non-petroleum based solvent;
f) continuing treatment of said extraction or dispersion system for such a time that sufficient high electric field intensity microwave energy has been absorbed by said biomass to induce the release of components making up said biomass at a level equivalent or superior to that observed under thermal equilibrium conditions.

10. The method as defined in claim 9 where a further substance is added to said extraction or dispersion system and more specifically into said multi-component biomass forming part of said extraction or dispersion system so as to further increase the localized heating into said multi-component biomass forming part of said extraction or dispersion system and to reduce the treatment time.

11. The method as defined in claim 9 including the step of recovering at least one component or product from said extraction or dispersion system after the equilibrium of said extraction or dispersion system has been preferentially shifted to said non-equilibrium state.

12. The method as defined in claim 9 where so produced extract or dispersion is used as the non-organic, non petroleum based solvent to further concentrate the quantity of components of biomass into said non-organic, non petroleum-based solvent.

13. The method as defined in claim 10 wherein the chemical susceptor is a compound of natural origin.

14. The method as defined in claim 10 wherein the chemical susceptor is a compound that does not interact with any of the substances making up said extraction or dispersion substance.

15. The method as defined in claim 14 where the chemical susceptor is an inorganic carbide.
